(19) United States
(12) Patent Application Publication
(10) Pub. No.: US 2023/0383356 A1
(43) Pub. Date: Nov. 30, 2023

(54) COMPOSITION FOR DIAGNOSING COLORECTAL CANCER, RECTAL CANCER OR COLORECTAL ADENOMA BY USING CPG METHYLATION CHANGE OF LINC01798 GENE, AND USE THEREOF

(71) Applicant: GENCURIX INC., Guro-gu, Seoul (KR)

(72) Inventors: Sang Rae CHO, Guro-gu, Seoul (KR); Young Ho MOON, Guro-gu, Seoul (KR); Jinil HAN, Guro-gu, Seoul (KR); Yun Young LEE, Guro-gu, Seoul (KR); Joon AN, Guro-gu, Seoul (KR)

(21) Appl. No.: 18/030,515

(22) PCT Filed: Oct. 8, 2021
(86) PCT No.: PCT/KR2021/013835

§ 371 Date: Apr. 6, 2023

(30) Foreign Application Priority Data
Oct. 8, 2020 (KR) 10-2020-0130539

(51) Int. Cl. C12Q 1/6886

(52) U.S. Cl. CPC C12Q 1/6886; C12Q 2600/154

(57) ABSTRACT

The present invention relates to a composition, a kit, a nucleic acid chip and a method, for diagnosing colorectal cancer, rectal cancer or colorectal adenoma by detecting the methylation level of CpG sites in a LINC01798 gene, in which colorectal cancer, rectal cancer, or colorectal adenoma can not only be diagnosed accurately and rapidly, but can also be diagnosed early.

### TECHNICAL FIELD

This application claims the priority of Korean Patent Application No. 10-2020-0130539, filed on Oct. 8, 2020, the entirety of which is a reference of the present application.

The present invention relates to a composition, kit, nucleic acid chip and method for diagnosing colorectal cancer, rectal cancer or colorectal adenoma by detecting methylation level of CpG region of LINC01798 gene.

### BACKGROUND ART

The large intestine is the last part of the digestive system, and is about 2 m long, and is divided into the cecum, ascending colon, transverse colon, descending colon, sigmoid colon, and rectum. Colorectal cancer is a cancer that occurs in this area and is mostly adenocarcinoma, and it is largely divided into colon cancer and rectal cancer by area.

Colorectal cancer can occur in any part of the large intestine or rectum, but it occurs most often in the rectum (about 40%), followed by about 30% in the S colon, which is adjacent to the rectum. Changes in dietary habits are significantly increasing the incidence and mortality of colorectal cancer in Korea, and it is also a major factor in cancer-related deaths in the United States and Europe (American Cancer Society statics for 2009).

The diagnosis of colorectal cancer is simply carried out by a fecal occult blood test during a health checkup, but additional examinations and tests are needed to actually confirm colorectal cancer. Although examinations are mainly performed through digital colonoscopy (barium enema), and colonoscopy including rectal examination, sigmoidoscopy, early detection of colorectal cancer patients is very important in increasing the survival rate of patients because the prognosis varies greatly depending on the progress of the cancer at the time of diagnosis.

On the other hand, epigenetics is a field that studies the regulation of gene expression in a state where the nucleotide sequence of DNA is not changed. Epigenetics studies the regulation of gene expression through epigenetic mutations such as DNA methylation, acetylation, methylation, phosphorylation and ubiquitination of miRNAs or histones.

Double DNA methylation is the most studied epigenetic mutation. Epigenetic mutation can lead to mutations in gene function and changes to tumor cells. Therefore, DNA methylation is associated with the expression (or suppression and induction) of disease-regulating genes in cells, and cancer diagnosis methods through DNA methylation measurement have recently been proposed. In particular, since cancer-specific methylation often occurs in advance even in pre-cancerous tissues, detection of cancer-specific methylation is highly likely to be used for cancer diagnosis.

Therefore, it is necessary to develop an effective colorectal cancer, colorectal cancer, or colorectal adenoma-specific methylation marker capable of predicting the risk of colorectal cancer and colorectal cancer or colorectal adenoma with similar characteristics.

### DISCLOSURE

### Technical Problem

Accordingly, the present inventors discovered that a specific gene CpG region in colorectal cancer, rectal cancer or colorectal adenoma was hypermethylated, and the present invention was completed by developing a composition, a kit, a nucleic acid chip and a method capable of diagnosing colorectal cancer, rectal cancer or colorectal adenoma by detecting the methylation level. completed the present invention.

Accordingly, an object of the present invention is to provide a composition for diagnosing colorectal cancer, rectal cancer or colorectal adenoma, comprising an agent for measuring the methylation level of the CpG region of a specific gene.

In addition, an object of the present invention is to provide a composition for diagnosing colorectal cancer, rectal cancer or colorectal adenoma, consisting of an agent for measuring the methylation level of the CpG region of a specific gene.

In addition, an object of the present invention is to provide a composition for diagnosing colorectal cancer, rectal cancer or colorectal adenoma, essentially consisting of an agent for measuring the methylation level of the CpG region of a specific gene.

In addition, another object of the present invention is to provide a kit for diagnosing colorectal cancer, rectal cancer or colorectal adenoma, comprising a PCR primer pair for amplifying a fragment containing a CpG region of a specific gene and a sequencing primer for pyrosequencing the PCR product amplified by the primer pair.

In addition, another object of the present invention is to provide a nucleic acid chip for diagnosing colorectal cancer, rectal cancer, or colorectal adenoma immobilized with a probe capable of hybridizing with a fragment containing a CpG region of a specific gene under stringent conditions.

In addition, another object of the present invention is to provide a method for providing information for diagnosing colorectal cancer, rectal cancer, or colorectal adenoma, comprising measuring, and comparing a methylation level of CpG region of a specific gene from different samples.

In addition, another object of the present invention is to use of an agent for measuring the methylation level of the CpG region of the LINC01798 gene for manufacturing a preparation for diagnosing colorectal cancer, rectal cancer, or colorectal adenoma.

In addition, another object of the present invention is to provide a method for diagnosing colon cancer, rectal cancer or colon adenoma comprising
a) obtaining a sample from a subject and measuring the methylation level of the CpG region of the LINC01798 gene; and
b) determining whether colorectal cancer, rectal cancer, or colorectal adenoma is present based on the measured methylation level.

### Technical Solution

In order to achieve the above object, the present invention provides a composition for diagnosing colorectal cancer, rectal cancer or colorectal adenoma comprising an agent for measuring the methylation level of the CpG region of the LINC01798 (long intergenic non-protein coding RNA 1798) gene.

In addition, the present invention provides a composition for diagnosing colorectal cancer, rectal cancer or colorectal adenoma consisting of an agent for measuring the methylation level of the CpG region of the LINC01798 (long intergenic non-protein coding RNA 1798) gene.

In addition, the present invention provides a composition for diagnosing colorectal cancer, rectal cancer or colorectal adenoma essentially consisting of an agent for measuring the methylation level of the CpG region of the LINC01798 (long intergenic non-protein coding RNA 1798) gene.

In order to achieve another object of the present invention, the present invention provides a kit for diagnosing colorectal cancer, rectal cancer or colorectal adenoma, comprising a pair of primers for amplifying a fragment containing the CpG region of the LINC01798 gene.

In addition, in order to achieve another object of the present invention, the present invention provides a nucleic acid chip for diagnosing colorectal cancer, rectal cancer or colorectal adenoma on which a probe that can hybridize with a fragment containing the CpG region of the LINC01798 gene is immobilized.

In addition, in order to achieve another object of the present invention, the present invention provides a method for providing information for the diagnosis of colorectal cancer, rectal cancer or colorectal adenoma, comprising measuring the methylation level of the CpG region of the LINC01798 gene from a sample of a patient suspected of having colorectal cancer, rectal cancer or colon adenoma; and
comparing the measured methylation level with the methylation level of the CpG region of the same gene in a normal control sample.

In addition, in order to achieve another object of the present invention, the present invention provides use of an agent for measuring the methylation level of the CpG region of the LINC01798 gene for manufacturing a preparation for diagnosing colorectal cancer, rectal cancer, or colorectal adenoma.

In addition, in order to achieve another object of the present invention, the present invention provides

a method for diagnosing colon cancer, rectal cancer or colon adenoma comprising
a) obtaining a sample from a subject and measuring the methylation level of the CpG region of the LINC01798 gene; and
b) determining whether colorectal cancer, rectal cancer, or colorectal adenoma is present based on the measured methylation level.

Unless defined otherwise, all technical and scientific terms used herein have the same meaning as commonly understood by one of ordinary skill in the art. The following references provide one of the skills with general definitions of several terms used in the specification of the present invention: Singleton et al., DICTIONARY OF MICROBIOLOGY AND MOLECULAR BIOLOTY (2th ed. 1994); THE CAMBRIDGE DICTIONARY OF SCIENCE AND TECHNOLOGY (Walkered., 1988); [symbol] Hale & Marham, THE HARPER COLLINS DICTIONARY OF BIOLOGY.

Hereinafter, the present invention will be described in detail.

The present invention provides a composition for diagnosing colorectal cancer, rectal cancer, or colorectal adenoma, comprising an agent for measuring the methylation level of CpG region of LINC01798 gene.

As used herein, the term “methylation” refers to the attachment of a methyl group to a base constituting DNA. Preferably, in the present invention, methylation means whether methylation occurs at a specific CpG region cytosine of a specific gene. When methylation occurs, the binding of transcription factors is hindered and the expression of specific genes is suppressed, conversely, when unmethylation or hypomethylation occurs, the expression of a specific gene increases.

In addition to A, C, G, and T, in mammalian cell genomic DNA, there is a fifth base called 5-methylcytosine (5-mC) with a methyl group attached to the fifth carbon of the cytosine ring. Methylation of 5-methylcytosine occurs only at the C of the CG dinucleotide (5′-mCG-3′), called CpG, and methylation of CpG inhibits the expression of alu or transposon and genomic repeats. In addition, since 5-mC of the CpG is easily deaminated naturally to become thymine (T), CpG is the region where most epigenetic changes occur frequently in mammalian cells.

As used herein, the term “measuring of methylation level” refers to measuring the methylation level of the CpG region of the LINC01798 gene, it can be measured through a detection method according to bisulfite treatment or a bisulfite-independent detection method. Measurement of methylation levels can be measured by methylation-specific PCR, such as methylation-specific polymerase chain reaction (MSP), real time methylation-specific polymerase chain reaction, PCR using methylated DNA-specific binding proteins, or quantitative PCR. Alternatively, it may be measured by automatic sequencing such as pyrosequencing and bisulfite sequencing, but is not limited thereto. In addition, it can be measured using a ten-eleven translocation protein (TET protein) as a bisulfite-independent detection method (See Nature Biotechnology, volume 37, pages 424-429 (2019)). The TET protein is an enzyme that acts on DNA and is involved in chemical changes of bases, unlike bisulfite treatment, all C except for methylated C are changed to T, only methylated C is changed to T in TET protein, enabling more efficient detection.

Preferably, the CpG region of the LINC01798 gene refers to a CpG region present on the DNA of the gene. The DNA of the gene is a concept that comprises all of a series of structural units necessary for the expression of the gene and operably linked to each other, and includes, for example, promoter regions, open reading frames (ORFs) and terminator regions. Therefore, the CpG region of the LINC01798 gene may exist in the promoter region, open reading frame (ORF) or terminator region of the gene.

Preferably, measuring the methylation level of the CpG region of the LINC01798 gene in the present invention may mean measuring the cytosine methylation level of the CpG region of the gene described in Table 1 below.

TABLE 1

| Symbol | Genome Build | Chromosome | Region |
|---|---|---|---|
| LINC01798 | GRCh37 | 2 | 66802891-66804831 |

In the present invention, the CpG site is located in a genetic region selected between +/−4000 base (4 kb) from the transcription start site (TSS) of the LINC01798 gene (NR_110156), in addition to LINC01798, it is located between 6.5 kb base from the transcription start site (TSS) of the MEIS1 gene (AK098174), preferably, the CpG region is located at the end of MEIS1 (Meis homeobox 1) gene and at the first intron region of LINC01798 gene.

In the present invention, the nucleotide sequence of the human genomic chromosomal region was expressed according to The February 2009 Human Reference Sequence (GRCh37), but the specific sequence of the human genomic chromosomal region may be slightly changed as the results of genome sequence research are updated, according to these changes, the expression of the chromosomal region of the human genome of the present invention may be different. Therefore, even if the expression of the human genomic chromosome region expressed according to The February 2009 Human Reference Sequence (GRCh 37) of the present invention is changed differently from now as the human reference sequence is updated after the filing date of the present invention, it will be apparent that the scope of the present invention extends to the altered human genomic chromosomal region. These changes can be easily recognized by anyone having ordinary knowledge in the art to which the present invention belongs.

In the present invention, the agent for measuring the methylation level of the CpG region may include a cytosine base-modifying compound or a methylation-sensitive restriction enzyme, primers specific for methylated allelic sequences of the LINC01798 gene, and primers specific for unmethylated allelic sequences.

The compound modifying the cytosine is a compound modifying unmethylated cytosine or methylated cytosine, may be bisulfite or a salt thereof that modifies unmethylated cytosine, preferably sodium bisulfite, or a TET protein that modifies methylated cytosine, but is not limited thereto. A method for detecting methylation of CpG regions by modifying such cytosine is well known in the art (WO01/26536; US2003/0148326A1).

In addition, the methylation-sensitive restriction enzyme is a restriction enzyme capable of specifically detecting methylation of a CpG region, and may be a restriction enzyme containing CG as a recognition site of the restriction enzyme. Examples include, but are not limited to, SmaI, SacII, EagI, HpaII, MspI, BssHII, BstUI, and NotI. Depending on methylation or unmethylation at C of the restriction enzyme recognition site, cleavage by the restriction enzyme varies, and this can be detected through PCR or Southern Blot analysis. Other methylation sensitive restriction enzymes other than the above restriction enzymes are well known in the art.

The primers may comprise primers specific for a methylated allelic sequence of the LINC01798 gene and primers specific for an unmethylated allelic sequence.

In the present invention, the term “primer” is a nucleic acid sequence having a short free 3′ terminal hydroxyl group that can form base pairs with a complementary template and a short nucleic acid sequence that serves as a starting point for copying the template strand. Primers can initiate DNA synthesis in the presence of reagents for polymerization (i.e., DNA polymerase or reverse transcriptase) and four different nucleoside triphosphates in an appropriate buffer solution and temperature. In addition, primers are sense and antisense nucleic acids with a sequence of 7 to 50 nucleotides, which may incorporate additional features that do not change the basic properties of the primers that serve as starting points for DNA synthesis.

The primers of the present invention may be preferably designed according to the sequence of a specific CpG region to be analyzed for methylation, more preferably, it may be at least one selected from the group consisting of a pair of primer capable of specifically amplifying cytosine that has been methylated and not modified by bisulfite, a pair of primer capable of specifically amplifying cytosine modified by bisulfite that is not methylated, a pair of primer capable of specifically amplifying methylated and modified cytosine by TET family proteins and a pair of primer capable of specifically amplifying cytosine that is not methylated and not modified by TET family proteins

Accordingly, the present invention provides a kit for diagnosing colorectal cancer, rectal cancer or colorectal adenoma, comprising a pair of primers for amplifying a fragment containing the CpG region of the LINC01798 gene.

In addition to the above agents, the composition and kit may further comprise polymerase agarose, a buffer solution required for electrophoresis, and the like.

In addition, the present invention provides a nucleic acid chip for diagnosing colorectal cancer, rectal cancer or colorectal adenoma on which a probe that can hybridize with a fragment containing the CpG region of the LINC01798 gene is immobilized.

In the present invention, the term “nucleic acid” refers to oligonucleotides, nucleotides, polynucleotides, or fragments thereof, single-stranded or double-stranded DNA of genomic origin or synthetic origin or RNA, DNA of genomic origin or of synthetic origin in the sense or antisense strand or RNA, peptide nucleic acid (PNA) or DNA or RNA amount materials of natural or synthetic origin. If the nucleic acid is RNA, it is apparent to those skilled in the art that ribonucleotides A, G, C and U are substituted for deoxynucleotides A, G, C and T, respectively.

Since methylation starts from the outside of a gene regulatory region and progresses inside, genes involved in cell transformation can be diagnosed at an early stage by detecting methylation at the outside of a regulatory region.

Accordingly, it is possible to early diagnose cells likely to form colorectal cancer, rectal cancer, or colorectal adenoma using the methylated gene marker. When a gene confirmed to be methylated in cancer cells is methylated in a cell that appears clinically or morphologically normal, cells that appear normal are cancerous in progress. Therefore, colorectal cancer, rectal cancer, or colorectal adenoma can be diagnosed at an early stage by confirming methylation of colorectal cancer, colorectal cancer, or colorectal adenoma-specific genes in normal-looking cells.

In addition, the present invention provides a method for providing information for the diagnosis of colorectal cancer, rectal cancer or colorectal adenoma, comprising measuring the methylation level of the CpG region of the LINC01798 gene from a sample of a patient suspected of having colorectal cancer, rectal cancer or colon adenoma; and

comparing the measured methylation level with the methylation level of the CpG region of the same gene in a normal control sample.

A method for measuring the methylation level may be selected from the group consisting of PCR, methylation specific PCR, real time methylation specific PCR, PCR using methylated DNA specific binding protein, methylation determination using methylation sensitive restriction enzyme, quantitative PCR, DNA chip, pyrosequencing and bisulfite sequencing, but is not limited thereto.

Specifically, the methylation-specific PCR method is a method in which, after treating sample DNA with bisulfite, different types of primers are designed and used depending on whether CpG dinucleotide is methylated or not as primers to perform PCR. If the primer binding site is methylated, PCR is performed using the methylated primer, and if it is not methylated, PCR is performed using the normal primer. That is, this is a method in which sample DNA is treated with bisulfite, PCR is performed using two types of primers at the same time, and the results are compared.

Real-time methylation-specific PCR is a conversion of the methylation-specific PCR method into a real-time measurement method, after treating genomic DNA with bisulfite, PCR primers corresponding to the case of methylation are designed, and real-time PCR is performed using these primers. At this time, there are two methods: detection using a TaqMan probe complementary to the amplified nucleotide sequence and detection using SYBR green. Therefore, real-time methylation-specific PCR can selectively quantify only methylated DNA. At this time, a standard curve is prepared using in vitro methylated DNA samples, for standardization, it is a method of quantitatively analyzing the degree of methylation by amplifying a gene without a 5′-CpG-3′ sequence in the sequence together as a negative control group.

In the method of measuring methylation using a methylation-sensitive restriction enzyme, the methylation-sensitive restriction enzyme uses CpG dinucleotide as an action site, when this site is methylated, it cannot function as an enzyme. Therefore, if the sample DNA is treated with a methylation-sensitive restriction enzyme and amplified by PCR to include the enzyme target site, in the case of methylation, restriction enzyme is not acted upon and PCR amplification is performed, however, since unmethylated normal regions are cleaved by restriction enzyme and are not PCR amplified, methylation of a specific DNA region can be measured.

In the PCR or DNA chip methods using methylated DNA-specific binding protein, when a protein that specifically binds only to methylated DNA is mixed with DNA, only methylated DNA can be selectively separated because the protein specifically binds only to methylated DNA.

After mixing genomic DNA with methylated DNA-specific binding protein, only methylated DNA is selectively separated. After amplifying these separated DNAs using PCR primers corresponding to intron regions, methylation is measured by agarose electrophoresis. In addition, methylation can be measured by quantitative PCR, methylated DNA separated by methylated DNA-specific binding protein can be labeled with a fluorescent dye and hybridized to a DNA chip on which complementary probes are integrated, thereby measuring methylation. The methylated DNA specific binding protein herein is not limited to MBD2bt.

In addition, bisulfite pyrosequencing of DNA treated with bisulfite is based on the following principle. When methylation occurs at the CpG dinucleotide region, 5-methylcytosine (5-mC) is formed, and this modified base is converted to uracil upon treatment with bisulfite. When DNA extracted from a sample is treated with bisulfite, if CpG dinucleotide is methylated, it is preserved as cytosine, and the remaining unmethylated cytosine is converted to uracil. Sequencing of the bisulfite-treated DNA can preferably be performed using a pyrosequencing method. A detailed description of pyrosequencing is known in the literature. [Ronaghi et al, Science 1998 Jul. 17, 281(5375), 363-365; Ronaghi et al, Analytical Biochemistry 1996 Nov. 1, 242(1), 84-9; Ronaghi et al. Analytical Biochemistry 2000 Nov. 15, 286 (2): 282-288; Nyr, P. Methods Mol Biology 2007, 373, 114].

On the other hand, with the bisulfite-independent detection method using TET protein, only C methylated using TET protein can be converted to T, thereby detecting the base of the methylation site (See LIU, Yibin, et al., Nature Biotechnology volume 37, pages 424-429 (2019)).

When methylation occurs at the CpG dinucleotide region and 5-methylcytosine (5-mC) is formed in cytosine, when processing TET (ten-eleven translocation) protein, if the CpG dinucleotide is methylated, it is changed to uracil, unmethylated cytosines are conserved. Sequencing of TET-treated DNA is not limited to pyrosequencing methods, it can be analyzed using methods such as methylation-sensitive PCR (MSP), microarray, and next generation sequencing (NGS).

Preferably, the method for providing information for diagnosing colorectal cancer, rectal cancer or colorectal adenoma of the present invention may be performed by a method characterized by comprising a) obtaining a sample from the subject; b) obtaining genomic DNA from the sample; c) treating the obtained genomic DNA with a compound that modifies unmethylated cytosine; d) obtaining a PCR product by amplifying the treated DNA by PCR using a primer capable of amplifying the CpG region of the LINC01798 gene; and e) measuring the degree of methylation of the PCR product.

The genomic DNA of the step b) may be obtained using a phenol/chloroform extraction method, SDS extraction method, CTAB separation method, or a commercially available DNA extraction kit commonly used in the art.

In the present invention, the term ‘sample’ refers to a wide range of body fluids, comprising all biological fluids obtained from individuals, body fluids, cell lines, tissue culture, etc., depending on the type of analysis to be performed. Methods for obtaining bodily fluid and tissue biopsies from mammals are commonly known, in the present invention, the sample may be preferably selected from the group consisting of human-derived materials including tissues, cells, blood, plasma, serum, feces and urine. Abnormal methylation changes in cancer tissue show considerable similarity to methylation changes in genomic DNA obtained from biological samples such as cells, whole blood, serum, plasma, saliva, sputum, cerebrospinal fluid or urine, when the marker of the present invention is used, there is an advantage in that it is possible to easily diagnose colorectal cancer, rectal cancer, or colorectal adenoma through blood or bodily fluid.

The present invention provides use of an agent for measuring the methylation level of the CpG region of the LINC01798 gene for manufacturing a preparation for diagnosing colorectal cancer, rectal cancer, or colorectal adenoma.

The present invention provides a method for diagnosing colon cancer, rectal cancer or colon adenoma comprising
a) obtaining a sample from a subject and measuring the methylation level of the CpG region of the LINC01798 gene; and
b) determining whether colorectal cancer, rectal cancer, or colorectal adenoma is present based on the measured methylation level.

In one embodiment, the present invention provides a method for diagnosing and treating colorectal cancer, rectal cancer or colorectal adenoma in an subject comprising:
i) obtaining a sample from the subject;
ii) measuring the methylation level of the CpG region of the LINC01798 gene from the sample;
iii) determining whether colorectal cancer, rectal cancer, or colorectal adenoma is present based on the measured methylation level; and
iv) administering a therapeutic drug for treating colorectal cancer, rectal cancer, or
colorectal adenoma to the determined subject, or treating colorectal cancer, rectal cancer, or colorectal adenoma through surgery.

Methods comprising steps i) to iv) are understood in accordance with the methods comprising steps a) to b) described above.

The step iv) is a step of treating the disease through means such as administration of therapeutic drugs or surgery to the subject diagnosed with the disease in the step iii).

The ‘treatment’ of the present invention comprehensively refers to improving colorectal cancer, rectal cancer or colorectal adenoma or symptoms of the disease, this may include curing, substantially preventing, or ameliorating the condition, and it includes, but is not limited to, alleviating, curing or preventing one or most of the symptoms resulting from colorectal cancer, rectal cancer or colorectal adenoma.

The type of the ‘therapeutic drug’ is not particularly limited as long as it is a type of drug commonly used for the treatment of colorectal cancer, rectal cancer or colorectal adenoma. In addition, the therapeutic drug is administered to the subject in a ‘therapeutically effective amount’, the therapeutically effective amount can be determined by those skilled in the art in consideration of various factors such as the age, body weight, health status, sex, disease severity, diet and excretion rate of the patient as well as the unique properties of the drug, the route of administration, and the number of treatments. The route of administration of the therapeutic drug is not particularly limited, it may be administered orally or parenterally, both topical as well as systemic routes of administration are included. The parenteral administration is not limited thereto, but may be, for example, intranasal drug application, subcutaneous injection, etc. another example may be to use a method such as intramuscular injection or intravenous injection.

The ‘sample’ of the present invention is obtained separately from a subject suspected of having a disease, but is not limited thereto, but may be selected from the group consisting of cells, tissue, blood, serum, plasma, saliva, sputum, mucosal fluid and urine, the ‘subject’ may be an animal, preferably an animal, including a mammal, particularly a human, and may be an animal-derived cell, tissue, organ, or the like. The subject may be a patient in need of the effect.

As used herein, the term “comprising” is used synonymously with “containing” or “being characterized”, and does not exclude additional ingredients or steps not mentioned in the composition or method. The term “consisting of” means excluding additional elements, steps, or ingredients not otherwise specified. The term “essentially consisting of” means including the mentioned elements or steps as well as any element or step that does not substantially affect basic characteristics of the mentioned elements or steps in the scope of compositions or methods.

### Advantageous Effects

As described above, since hypermethylation of the CpG region of the LINC01798 gene is specifically shown in colorectal cancer, rectal cancer or colorectal adenoma, using the composition, kit, chip or method according to the present invention, colorectal cancer, rectal cancer, or colorectal adenoma can be diagnosed accurately and quickly, and can be diagnosed at an early stage.

### DESCRIPTION OF DRAWINGS

FIG. 1 is a result of confirming the methylation information of the LINC01798 gene in a total of 32 cancer types.

FIG. 2 is a result confirming the colorectal cancer diagnosis accuracy of the LINC01798 gene selected according to the present invention.

FIG. 3 is a result of confirming the difference in methylation between a colorectal cancer tumor tissue (tumor) cell line group and a non-colorectal cancer tumor tissue (others) cell line group.

FIG. 4 is a result of confirming the methylation differences in tumor tissue (cancer), colorectal adenoma tissue (adenoma), and normal tissue (normal) in colorectal adenoma.

FIG. 5 is a result of confirming the qMSP-based methylation differences in tumor tissue (cancer) and normal tissue (non-tumor) surrounding the tumor in adenoma of the colon.

FIG. 6 is a comparative example, and is a result of confirming the methylation information of the OPLAH gene.

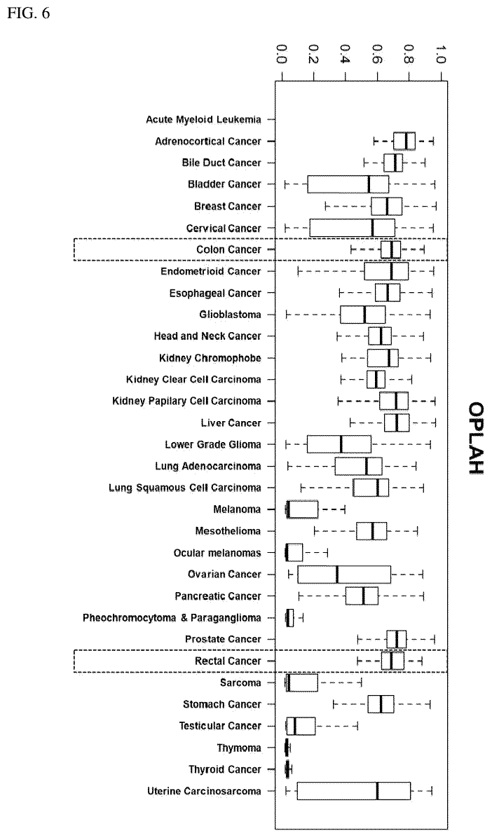

### MODES FOR THE INVENTION

Hereinafter, the present invention will be described in detail.

However, the following Examples are just illustrative of the present invention, and the contents of the present invention are not limited to the following Examples.

### Example 1: Screening for Colorectal Cancer-Specific Methylation Genes

In order to select methylation genes specifically found in colorectal cancer, a large-scale methylation comparison study was conducted between cancer tissues obtained from cancer surgery of colorectal cancer patients and normal tissues using two large-scale methylation microarray chip data (See Table 2). Tumor tissue used in this study refers to cancer tissue of colorectal cancer, and non-tumor tissue refers to tissue other than cancer tissue including normal tissue.

TABLE 2

| | dataset#1 | dataset#2 | Total |
|---|---|---|---|
| tumor | 112 | 395 | 507 |
| non-tumor | 149 | 45 | 194 |

In order to select colorectal cancer-specific methylation genes, DNA was extracted from each tissue, and the degree of methylation of the gene region was confirmed using Infinium Human Methylation 450 Beadchip microarray.

DNA extracted from each tissue is converted through bisulfite treatment. Through this, the cytosine is modified depending on whether the DNA region is methylated or not. The Probes used in the microarray experiment were specifically designed for methylation and unmethylation to determine whether cytosine were modified at gene methylation region.

The microarray experiment measures the degree of methylation of genes through about 450,000 (450 k) probes, each representing the methylation region of a gene, the result of each probe derived through the experiment is presented as a beta value. The beta value ranges from 0 to 1, and the closer to 1, the higher the degree of methylation of the corresponding genetic region.

To identify differentially methylated regions (DMRs) between the tumor and non-tumor groups, the empirical Bayes t-test, the Limma (Linear Models for Microarray Data) method, was used to identify gene regions showing statistically significant methylation differences between groups.

The Limma method is known to be least affected by outliers among several methylation statistical analysis methods that identify differences between groups. Therefore, it is less affected by abnormal measurement values of some samples and is suitable for finding cancer-specific markers. In this experiment, it was judged that there was a significant difference in methylation between the two groups as the adjusted p-value derived through the Limma method decreased.

In particular, to search for tumor-specific methylation regions, regions with higher methylation in tumor tissue than in non-tumor tissue among gene regions with significant differences in beta values between tumor and non-tumor groups were selected as cancer-specific biomarker candidates.

As a result of Limma analysis in each of the two datasets, having a significantly lower p-value (Top 10% with the lowest P value) when comparing tumor groups compared to non-tumor groups, and gene regions showing a large difference of beta value of 0.2 or more between groups were selected as tumor-specific hypermethyalted regions. Through this, among about 450,000 genetic regions, 3,878 genetic regions showing tumor-specific hypermethylation in common in all datasets were selected as biomarker candidates.

### Example 2: Screening for Colorectal Cancer-Specific Hypermethylation Genes

In the genetic regions of the 3,878 biomarkers identified in Example 1, in tumors other than colorectal cancer, the methylation level of each corresponding region was confirmed and compared to find a genetic region specific to colorectal cancer or colorectal adenoma among biomarkers. By analyzing the results of the DNA methylation 450 k array experiment of The Cancer Genome Atlas (TCGA), a public cancer gene database, methylation information of genetic regions corresponding to 32 cancer types was confirmed. Among them, as a result of confirming the genetic region that showed a significantly higher beta value in colorectal cancer, rectal cancer or colorectal adenoma compared to the remaining 30 types of cancers other than colorectal cancer and rectal cancer, it was confirmed that the genetic region of the LINC01798 gene had colorectal cancer, rectal cancer, or colorectal adenoma-specific methylation among the 3,878 genetic regions.

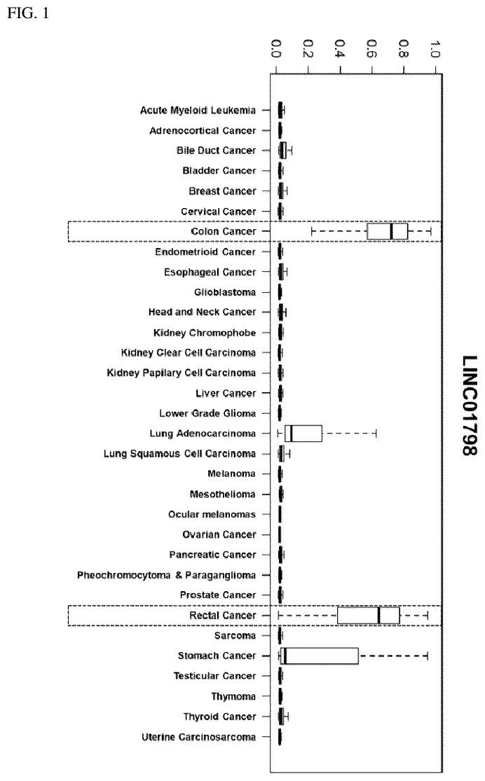

The degree of methylation of the gene through the microarray experiment for tumor tissue (cancer tissue of colorectal cancer) and non-tumor tissue (tissue other than cancer tissue including normal tissue) for the gene is shown in FIG. 1. As for the degree of methylation, the result of each probe derived through the test was expressed as a beta value, the beta value ranges from 0 to 1, and the closer to 1, the higher the methylation level of the corresponding genetic region.

On the other hand, in the case of genetic regions where methylation differences are observed when comparing tumor tissue and non-tumor tissue of colorectal cancer, rectal cancer, or colorectal adenoma, methylation may also occur in cancers other than colorectal cancer, rectal cancer or colorectal adenoma. That is, methylation specific to colorectal cancer, rectal cancer, or colorectal adenoma was not confirmed.

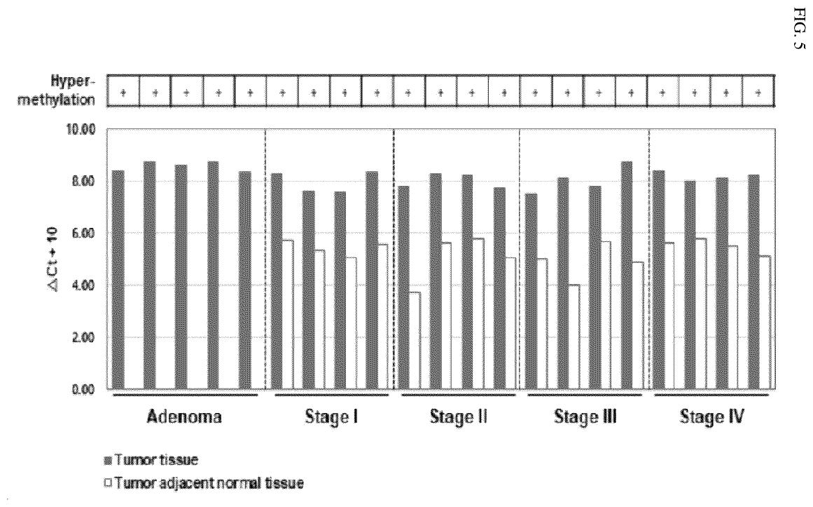

For example, in the case of the OPLAH (5-oxoprolinase, ATP-hydrolysing) gene, among the 3,878 genetic regions identified in Example 1, it was one of the regions where the largest difference in methylation between tumor tissue and non-tumor tissue was identified. As shown in FIG. 5, it was confirmed that high methylation occurred in all types of carcinomas except Acute Myeloid Leukemia, Ocular melanomas, Pheochromocytoma & paraganglioma, Thymoma, Thyroid cancer.

The 32 types of cancer are as follows: Acute Myeloid Leukemia, Adrenocortical Cancer, Bile Duct Cancer, Breast Cancer, Cervical Cancer, Colon Cancer, Endometrioid Cancer Cancer, Esophageal cancer, Glioblastoma, Head and neck cancer, Kidney chromophobe, Kidney clear cell carcinoma, Kidney papillary cell carcinoma Papillary cell carcinoma, Liver cancer, Lower grade glioma, Lung adenocarcinoma, Melanoma, Mesothelioma, Ocular melanomas, Ovarian cancer cancer, Pancreatic cancer, Pheochromocytoma & paraganglioma, Prostate cancer, Rectal cancer, Sarcoma, Stomach cancer, Testicular cancer, Thymoma, Thyroid cancer, Uterine carcinosarcoma.

Among these genetic regions, it is not a pseudogene, and the corresponding region exists in the CpG island region and is located in a genetic region selected between +/−4000 bases (4 kb) from the transcription start site (TSS) of the gene, when present on an autosome, it was selected as a colorectal cancer-specific hypermethylated gene. As a result, as shown in Table 3 below, one gene was selected (see FIG. 1).

TABLE 3

| | | Location | |
|---|---|---|---|
| Symbol | Name | (Chromosome) | CpG Island |
| LINC01798 | Long Intergenic Non- | 2 | Island |
| | protein Coding RNA 1798 | | |

### Example 3: Confirmation of Colorectal Cancer Specificity of Selected Genes in Cell Lines

To confirm that the selected LINC01798 gene exhibits colorectal and rectal cancer-specific methylation that is distinct from other cancers, methylation patterns in 1,022 cancer cell lines derived from 14 tissues were analyzed using a public database. The data is the result of experiment DNA extracted from each cell line on the Infinium Human Methylation 450 Beadchip microarray according to the manufacturer's standardized methylation analysis experiment procedure.

As the result of the experiment, the degree of gene methylation is measured through about 450,000 probes as in Example 1, and the methylation value of each probe is presented as a beta value. The beta value ranges from 0 to 1, and the closer to 1, the higher the degree of methylation of the corresponding genetic region.

The 14 tissues are as follows: aerodigestive tract, blood, bone, breast, digestive system, kidney, lung, nervous system, pancreas, skin, soft tissue, thyroid, urogenital system, and other tissues.

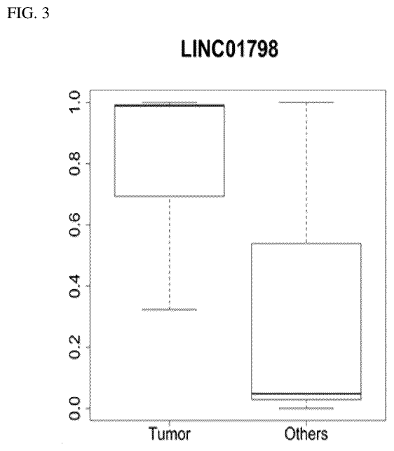

To confirm colorectal cancer, rectal cancer or colorectal adenoma-specific methylation of the selected LINC01798 gene, methylation data derived from 1,022 cell lines were largely classified into colorectal cancer cell line groups (n=51) and non-colorectal cancer cell line groups (n=971). As shown in FIG. 3, it was confirmed that the colorectal cancer cell line group exhibited a higher methylation value than the non-colorectal cancer cell line group.

To identify differentially methylated regions (DMRs) between the two groups, the Limma (Linear Models for Microarray Data) method, an empirical Bayes t-test, was used to identify gene regions showing statistically significant methylation differences between groups.

TABLE 4
Differences in methylation between the colorectal
cancer cell line group and the non-colorectal cancer
cell line group of the selected LINC01798 gene

| Symbol | Difference (average Δβ) | adjusted p-value |
|---|---|---|
| LINC01798 | 0.53 | 1.54e−22 |

In the analysis using cell lines, it was confirmed that the LINC01798 gene was colorectal cancer-specific, as it had a significantly lower adjusted p-value in colorectal cancer and rectal cancer cell lines than in other cancer cell lines.

### Example 4: Diagnostic Performance Evaluation of Colorectal Cancer, Rectal Cancer or Colorectal Adenoma Diagnostic Marker Candidates

In order to confirm the usefulness of the selected gene as a diagnostic marker in colorectal cancer, the accuracy of colorectal cancer diagnosis according to the degree of methylation was evaluated.

To evaluate the accuracy of diagnosis, sensitivity and specificity are used. Through the calculation of sensitivity and specificity values for possible cut-off values of continuous diagnostic test measurements, a ROC (Receiver Operating Characteristic) curve presenting changes in sensitivity and specificity according to cut-off values can be displayed. Diagnostic accuracy can be measured by the area under the ROC curve (AUC). The AUC value has a value between 0.5 and 1, and the higher the value, the higher the diagnostic accuracy. If the AUC value is 1, it means that the diagnosis result is a perfectly accurate test, but if it is 0.5, it is judged to be the same as a random result.

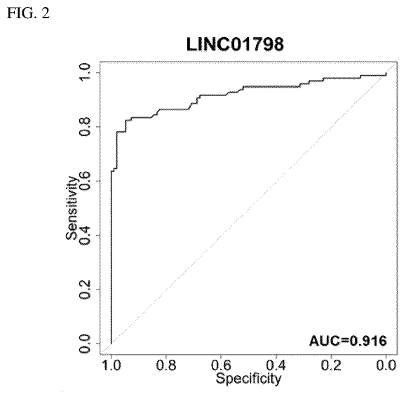

Results of analyzing the accuracy of cancer classification according to the degree of methylation between non-tumor and tumor tissues using selected genes using the collected methylation dataset, as shown in FIG. 2, all selected genes had an AUC (Area Under Curve) value of 0.900 or more, showing high diagnostic accuracy, confirming that the selected genes are useful for diagnosing colorectal cancer.

### Example 5: Confirmation of Methylation in Adenoma of Selected Genes

Adenoma is a pre-stage disease that progresses to colorectal cancer, and most colorectal cancers arise from adenomas. Therefore, early detection of adenoma is essential for early diagnosis of colorectal cancer. In order to confirm that hypermethylation biomarkers selected through previous studies show characteristics of hypermethylation in adenomas, the hypermethylation characteristics of genes selected from 64 colorectal cancer tissues, 42 colorectal adenoma tissues, and 41 non-tumor tissues were investigated.

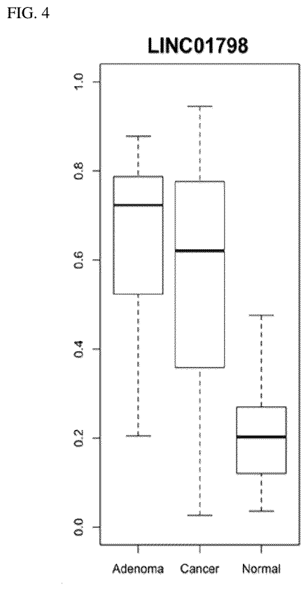

As a result of analyzing the methylation data derived from the Human Methylation450 Beadchip microarray experiment, as shown in FIG. 4, it was confirmed that the selected genes showed the same characteristics of hypermethylation significant with non-tumor tissue in colorectal adenoma as well as in colorectal cancer.

As a result, it was found that the selected gene can be used for diagnosis of colorectal adenoma as well as colorectal cancer.

### Example 6: qMSP-Based Methylation Measurement in Tissues of Selected Genes

In order to confirm colorectal cancer and adenoma-specific methylation of the LINC01798 gene in cancer tissues, quantitative methylation specific PCR (qMSP) techniques were used to measure methylation differences between cancer and non-cancer tissues. To this end, genomic DNA was isolated from cancer tissue and cancer tissue pairs of 16 colorectal cancer patients and cancer tissues of 5 adenoma patients, after treatment with bisulfite, the degree of amplification and methylation of the LINC01798-specific gene region was observed according to the generalized qMSP experiments method.

In addition, it specifically binds to and amplifies the genetic region modified with bisulfite, and the ACTB gene unrelated to methylation was used to standardize the amplified value of the region.

The methylation level obtained by amplifying the bisulfite-converted DNA by PCR is expressed as ΔCt+10, which is a value corrected by the cycle of threshold (Ct) value of ACTB used as an internal control. ΔCt+10 is defined as:

ΔCt+10=(Ct value of ACTB gene−Ct value of gene to be detected)+10

As shown in FIG. 5, the methylation of the LINC01798 gene has a relatively high ΔCt+10 value in colorectal cancer tissues regardless of stage compared to normal tissues around cancer, in particular, it was confirmed that the LINC01798 gene was hypermethylated in colorectal cancer and colorectal adenoma by showing a very high ΔCt+10 value in adenoma, a precancerous stage. This result actually shows that methylation of the selected LINC01798 gene is effective as a biomarker for diagnosis of colorectal cancer, especially early diagnosis.

As a result, it was found that the selected gene can be used for diagnosis of colorectal adenoma as well as colorectal cancer.

### INDUSTRIAL APPLICABILITY

As described above, since hypermethylation of the CpG region of the LINC01798 gene is specifically found in colorectal cancer, rectal cancer or colorectal adenoma, colorectal cancer, rectal cancer or colon adenoma can be diagnosed accurately and quickly as well as early by using the composition, kit, chip or method according to the present invention.

What is claimed is:

1. A composition for diagnosing colorectal cancer, rectal cancer, or colorectal adenoma, comprising an agent for measuring the methylation level of CpG region of LINC01798 gene.

2. The composition according to claim 1, wherein the CpG region is located between +/−4000 base pairs (4 kb) from the transcription start site of the gene.

3. The composition according to claim 1, wherein the agent for measuring the methylation level of CpG region of the gene is selected form the group consisting of compounds that modify unmethylated cytosine or methylated cytosine bases;
primers specific for methylated sequences of the CpG region of the LINC01798 gene; and
primers specific for unmethylated sequences.

4. The composition according to claim 3, wherein the compounds that modify the unmethylated cytosine base are bisulfite, a salt thereof, and the compounds that modify the methylated cytosine base are TET protein.

5. A kit for diagnosing colorectal cancer, rectal cancer or colorectal adenoma, comprising a pair of primers for amplifying a fragment containing the CpG region of the LINC01798 gene.

6. A nucleic acid chip for diagnosing colorectal cancer, rectal cancer or colorectal adenoma on which a probe that can hybridize with a fragment containing the CpG region of the LINC01798 gene is immobilized.

7. A method for providing information for the diagnosis of colorectal cancer, rectal cancer or colorectal adenoma, comprising measuring the methylation level of the CpG region of the LINC01798 gene from a sample of a patient suspected of having colorectal cancer, rectal cancer or colon adenoma; and
comparing the measured methylation level with the methylation level of the CpG region of the same gene in a normal control sample.

8. The method according to claim 7, wherein the method for measuring the methylation level is selected from the group consisting of bisulfite-free detection method, methylation-specific polymerase chain reaction, real time methylation-specific polymerase chain reaction, PCR using methylated DNA-specific binding proteins, quantitative PCR, pyrosequencing and bisulfite sequencing.

9. The method according to claim 7, wherein the sample is selected from the group consisting of tissue, cell, blood, plasma, serum, feces and urine.

10. Use of an agent for measuring the methylation level of the CpG region of the LINC01798 gene for manufacturing a preparation for diagnosing colorectal cancer, rectal cancer, or colorectal adenoma.

11. The use according to claim 10, wherein the CpG region is located between +/−4000 bases (4 kb) from the transcription start site of the gene.

12. The use according to claim 10, wherein the agent for measuring the methylation level of CpG region of the gene is selected form the group consisting of compounds that modify unmethylated cytosine or methylated cytosine bases;
primers specific for the methylated sequences of the CpG region of the LINC01798 gene; and
primers specific for unmethylated sequences.

13. A method for diagnosing colon cancer, rectal cancer or colon adenoma comprising
a) obtaining a sample from a subject and measuring the methylation level of the CpG region of the LINC01798 gene; and
b) determining whether colorectal cancer, rectal cancer, or colorectal adenoma is present based on the measured methylation level.

14. The method according to claim 13, wherein the method for measuring the methylation level is selected from the group consisting of bisulfite-free detection method, methylation-specific polymerase chain reaction, real time methylation-specific polymerase chain reaction, PCR using methylated DNA-specific binding proteins, quantitative PCR, pyrosequencing and bisulfite sequencing.

15. The method according to claim 10, wherein the sample is selected from the group consisting of tissue, cell, blood, plasma, serum, feces and urine.
